(19) United States
(12) Patent Application Publication
(10) Pub. No.: US 2019/0167491 A1
(43) Pub. Date: Jun. 6, 2019

(54) Wearable Absorbent Undergarment and Related Methods

(71) Applicant: Raymond Humes, La Puente, CA (US)

(72) Inventor: Raymond Humes, La Puente, CA (US)

(21) Appl. No.: 15/833,988

(22) Filed: Dec. 6, 2017

(51) Int. Cl. A61F 13/491; A61F 13/494; A61F 13/49; A61F 13/58; A61F 13/62; A61F 13/56; A61F 13/531; A61L 15/24; A61L 15/58

(52) U.S. Cl. CPC A61F 13/4915; A61F 13/49473; A61F 13/49007; A61F 13/58; A61F 13/622; A61F 2013/49046; A61F 13/531; A61L 15/24; A61L 15/58; A61F 2013/4948; A61F 13/5622

(57) ABSTRACT

A wearable absorbent undergarment designed to eliminate the presence of moisture on a wearer's skin due to urination or urinary incontinence. Additional inner layers with an orifice for containing the male penis occludes the penis in an internal cavity, and, thereby, prevents skin contact with expelled fluids. The isolation decreases the chance of irritation, discomfort, and diaper rash developments.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is generally in the field of health care. More specifically, the disclosed invention is in the field of geriatric care, child care, and patient care.

### Background of the Invention

Urinary incontinence is the involuntary loss of urine. The prevalence of urinary incontinence amongst adult males increases with age; however, urinary incontinence effects people of all ages as it can be a symptom of various medical issues such as nerve problems or prostate enlargement. Any disease, condition, or injury that damages nerves, such as spinal cord injuries, can lead to urination problems, and the constriction of the urethra due to prostate enlargement likewise affects the urinary system. (See Urinary Incontinence in Men, National Kidney and Urological Diseases Information Clearinghouse). The use of absorbent diapers, or incontinence pads, allows adults experiencing incontinency issues to continue daily activity without the encumbrance of dedicating immediate attention to uncontrollable urinary loss.

Moreover, young children also commonly wear diapers to help absorb urine when they are not yet toilet trained or when they are in the process of toilet training.

The design of modern diapers have a layered construction that transfers and distributes urine to an absorbent core structure with typical layers, including an outer shell, an inner absorbent layer, and a layer nearest the skin. These disposable diapers include a matrix of fluff, composed from wood pulp, and chemical crystals known as Super Absorbent Polymers (SAP). The role of the fluff is to distribute fluid, wherein the SAP is intended to absorb the fluid and contain it with in the core; however, issues arise from the natural limit to the absorbency of these materials and the slower rate of absorption relative to the rate of expulsion resulting in lingering contact with the skin. As a result, extra moisture left over from urination touches the skin and fluid may be squeezed from the absorbent layer and back onto the wearer's skin. This prolonged contact leads to discomfort and a condition known as diaper rash—a condition that causes skin in contact with the moisture to become red and inflamed. This tender area is then susceptible to blistering and infection.

Thus, a need exists for an absorbent undergarments that more efficiently protects the skin of the wearer from prolonged exposure to moisture from urination in a diaper or other type of underwear.

### SUMMARY OF THE INVENTION

In view of the foregoing, an object of this specification is to disclose a wearable absorbent undergarment with a modified inner layer that isolates the male genitalia to prevent skin exposure to expelled urine.

Another objective of the invention is to provide an improved wearable absorbent undergarment that does not significantly alter the familiar preexisting structures in use and does not require the addition of significantly more materials.

Another objective of the invention is to provide an absorbent undergarment comprising an additional layer overlaying the traditional three layers of the common disposable diaper. The placement of this additional layer creates a hollow cavity which encompasses the body of the penis, the isolation of which prevents bodily contact with expelled fluids.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is an orthogonal view of the wearable absorbent undergarment in an open configuration from above;

FIG. 2 is a perspective view of the wearable absorbent undergarment in an open configuration;

FIG. 3 is a cross-section view of the wearable absorbent undergarment; and,

FIG. 4 is a cross-section view of an alternative embodiment of the wearable absorbent undergarment.

In the figures, the following items correspond to the associated reference numerals:
Diaper—1000;
Inner top layer—2000;
Inner Absorbent Core—2010;
Inner bottom layer—2020;
Absorbent Core—3000;
Outer Layer—4000;
Orifice—5000;
Internal Cavity—6000;
Secondary layer—7000; and
Closures—8000.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

Disclosed is a wearable absorbent undergarment (i.e., a diaper) with an internal cavity to effectively contain and separate expelled bodily fluids from the body's skin. Further disclosed is a method of isolating male genitalia from the body by insertion of genitalia into the internal cavity through an orifice. The more specific details of the disclosed absorbent undergarment are described in connection with the figures.

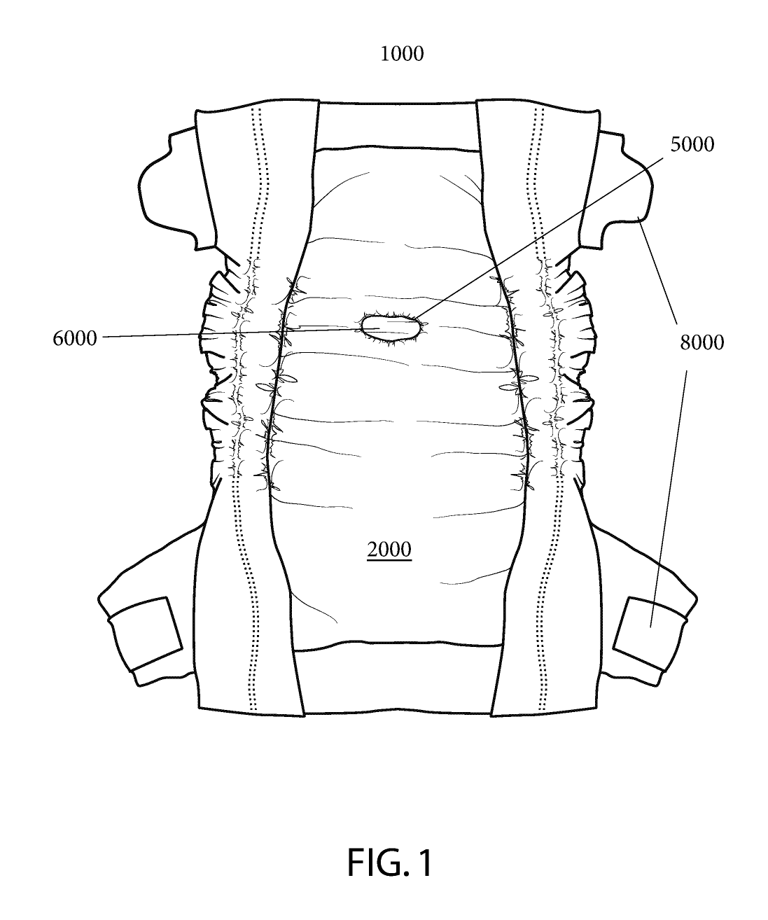

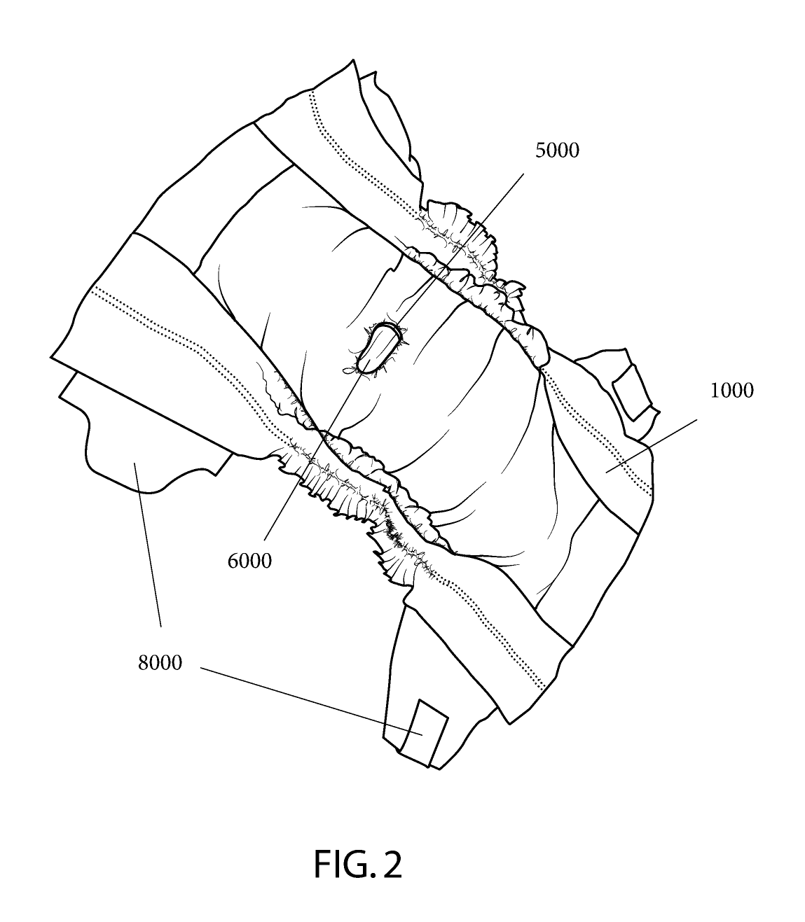

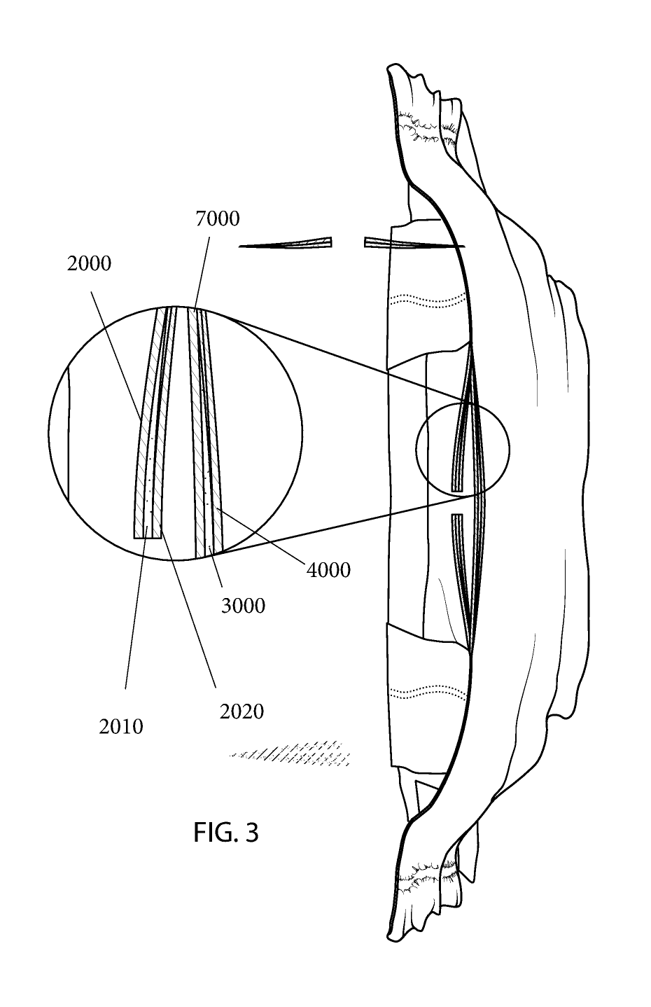

FIG. 1 is an orthogonal view of the wearable absorbent undergarment in an open configuration from above. FIG. 2 is a perspective view of the wearable absorbent undergarment in an open configuration. Referring to FIGS. 1 and 2, the inner top layer 2000 of the diaper 1000 comprises the nearest layer of the diaper that is in contact with the skin of the wearer. The inner top layer 2000 exhibits an orifice 5000 that transverses the inner top layer 2000, the inner absorbent core 2010 (see FIG. 3), and the inner bottom layer 2020 (see FIG. 3) and provides an entrance to the internal cavity 6000. As shown in the figures, the orifice 5000 is generally in a circular shape to conform to and encompass the shape of the penis in a form-fitting and ergonomic manner. In one embodiment, the orifice 5000 may be elastic around the perimeter opening. In another embodiment, the orifice 5000 may feature wing-shaped tabs to facilitate stretching and placement of the orifice 5000 around the wearer's penis. In an alternative embodiment, the orifice 5000 may be configured to encompass a multitude of shapes and may feature elements known to one of skill in the art to facilitate the placement of the orifice 5000 around the wearer's penis. In a typical embodiment, the inner layer 2000 is comprised of a stretchable, hydrophilic material that draws moisture away from the skin. In a preferred embodiment, the stretchable material of the inner top layer 2000 is in contact with the inner absorbent core 2010, which is in contact with the inner bottom layer 2020. The inner layers 2000, 2010, and 2020 are loosely affixed to the secondary layer 7000 (see FIG. 3) allowing for the inner top layer 2000 and, subsequently, the orifice 5000 to be pulled laterally to properly align with the varying natural anatomies of the wearers. Still referring to FIGS. 1 and 2, the undergarment features closures 8000 that allow the user to secure the undergarment to his waist. The closures 8000 may feature adhesives or a hood and loop fastening system. In use, a user would put the undergarment on and wrap the closures 8000 around his waist until it attaches with the corresponding fastening component on the other side of the undergarment.

FIG. 3 is a cross section of the diaper 1000 showing the inner top layer 2000, inner absorbency core 2010, inner bottom layer 2020, secondary layer 7000, absorbent core 3000, outer layer 4000, and internal cavity 6000. The inner top layer 2000 lays on top of the inner absorbency core 2010, which is sandwiched between the inner top layer 2000 and the inner bottom layer 2020. The inner layers 2000, 2010, and 2020 are configured to be above the secondary layer 7000, and, when worn, the inner top layer 2000 is in contact with the skin of the wearer. In one embodiment, the inner top layer 2000 is waterproof with some absorbency. The layered positions of the inner layers 2000, 2010, 2020 and the secondary layer 7000 create the internal cavity 6000. The orifice 5000 transverses the inner top layer 2000, inner absorbency core 2010, and inner bottom layer 2020 and provides an opening through which the male penis can be inserted into the internal cavity 6000, which occludes the penis from the body. The absorbent core 3000 is enclosed between the secondary layer 7000 and the outer layer 4000. The internal cavity 6000 is a pocket created by the stacking configuration of the inner layer 2000 and outer layer 4000. The inner layer 2000, secondary layer 7000 and outer layer 4000 are all attached at the outer sides of the undergarment akin to a traditional diaper. In a preferred embodiment, the inner absorbent core 2010 and the absorbent core 3000 may be comprised of superabsorbent polymers (SAPs), such has hydrogel, sodium polyacrylate, or polyacrylate absorbents. In a preferred embodiment, the undergarment is not bulky and may be the size of a standard diaper. In a preferred embodiment, the thickness of the inner absorbent core 2010 and the absorbent core 3000 is about half that of a traditional absorbent core in a traditional diaper, wherein the thickness of the inner absorbent core 2010 and the absorbent core 3000 is about the same thickness as the absorbent core in a traditional diaper.

In use, a wearer would unfold the undergarment and place it between the legs with the inner top layer 2000 closest to the body. The inner top layer 2000 and orifice 5000 would then be adjusted to align with the natural anatomy of the wearer and receive the penis and securing it within the internal cavity 6000, wherein the penis is housed within the internal cavity 6000 of the undergarment with an inner bottom layer 2020 in contact with the penis, wherein the inner layers 2000, 2010, and 2020 are between the penis and the rest of the body. The distal ends of the outer layer 4000 would then be joined at the wearer's waist via the closures 8000, encompassing the hips of the wearer and securing the diaper 1000 to the body.

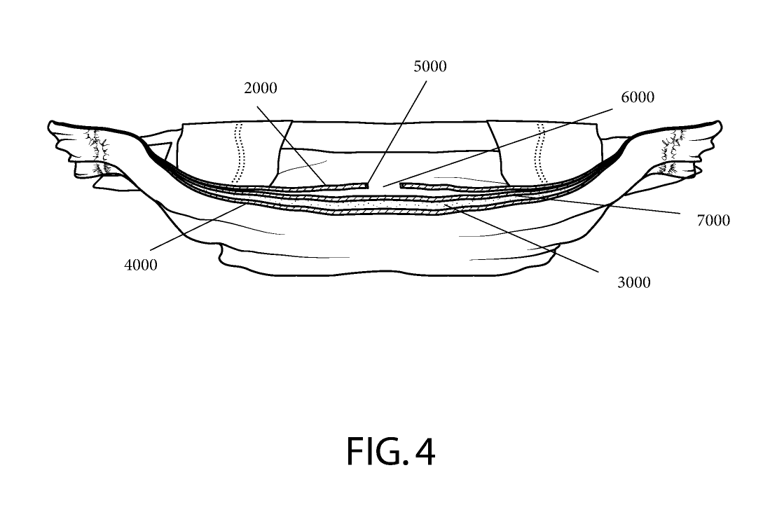

FIG. 4 is a cross-sectional view of an alternative embodiment of the wearable absorbent undergarment. In this alternative embodiment, the undergarment may be simplified, wherein the inner absorbent core 2010 (see FIG. 3) and inner bottom layer 2020 (see FIG. 3) may be omitted, wherein the undergarment features a water proof inner top layer 2000 with the orifice 5000.

The benefit of having the inner layers 2000, 2010, 2020, orifice 5000, and internal cavity 6000, is that it allows the user to occlude the penis from the rest of the body to prevent urine from touching the user's body.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A multi-layered absorbent undergarment with an inner top layer, an inner absorbent core, and an inner bottom layer, wherein the inner top layer, inner absorbent core, and inner bottom layer feature an orifice, whereby there is a layer of waterproof and semi-absorbent material between a user's genitals and body so that urine does not come into contact with the user's body during urinary incontinence.

2. A multi-layered absorbent undergarment comprising:
an inner top layer;
an inner absorbent core below the inner top layer;
an inner bottom layer below the inner absorbent core;
an orifice that is centrally disposed through the inner top layer, inner absorbent core, and inner bottom layer of the undergarment;
an internal cavity that lies beneath the inner bottom layer, whereby the genitals are placed through the orifice and housed;
a secondary layer that is beneath the internal cavity and affixed on the sides of the under garment to the inner bottom layer;
an outer layer that is affixed to the secondary layer of the undergarment;
an absorbent core configured to be disposed between the secondary layer and the outer layer; and,
at least one closure on the distal end of the undergarment, whereby the undergarment may be secured to a user.

3. The undergarment of claim 1, wherein the inner absorbent core is comprised of hydrogel.

4. The undergarment of claim 1, wherein the inner absorbent core is comprised of sodium polyacrylate.

5. The undergarment of claim 1, wherein the inner absorbent core is comprised of polyacrylate absorbents.

6. The undergarment of claim 1, wherein the perimeter of the orifice features elastic.

7. The undergarment of claim 1, wherein the orifice is further defined by winged tabs.

8. The undergarment of claim 1, wherein the closure is comprised of an adhesive.

9. The undergarment of claim 1, wherein the closure is further defined by a hook and loop fastening system.

10. The undergarment of claim 1, wherein the inner top layer is comprised of a hydrophilic material.

11. A method of keeping urine away from a user's body during urinary incontinence comprising the steps of:
obtaining an absorbent undergarment comprising an inner top layer, an inner absorbent core below the inner top layer, an inner bottom layer below the inner absorbent core, an orifice that is centrally disposed through the inner top layer, inner absorbent core, and inner bottom layer of the undergarment, an internal cavity that lies beneath the inner bottom layer, whereby the genitals are placed through the orifice and housed, a secondary layer that is beneath the internal cavity and affixed on the sides of the under garment to the inner bottom layer, an outer layer that is affixed to the secondary layer of the undergarment, an absorbent core configured to be disposed between the secondary layer and the outer layer, and, at least one closure on the distal end of the undergarment, whereby the undergarment may be secured to a user;
placing a user's genitals through the orifice and into the internal cavity of the orifice;
fixing one closure on one side of a user's waist; and,
fixing another closure on the other side of the user's waist.

12. The method of claim 11, wherein the user urinates into the internal cavity of the undergarment, whereby the urine does not come into contact with the user's body.

13. The method of claim 11, wherein the inner absorbent core is comprised of hydrogel.

14. The method of claim 11, wherein the inner absorbent core is comprised of sodium polyacrylate.

15. The method of claim 11, wherein the inner absorbent core is comprised of polyacrylate absorbents.

16. The method of claim 11, wherein the perimeter of the orifice features elastic.

17. The method of claim 11, wherein the orifice is further defined by winged tabs.

18. The method of claim 11, wherein the closure is comprised of an adhesive.

19. The method of claim 11, wherein the closure is further defined by a hook and loop fastening system.

20. The method of claim 11, wherein the inner top layer is comprised of a hydrophilic material.
